(19) United States
(12) Patent Application Publication
(10) Pub. No.: US 2012/0178825 A1
(43) Pub. Date: Jul. 12, 2012

(54) CYCLOHEXANEDIMETHANOL-BASED HYDROPHILIC POLYURETHANE UREA

(75) Inventors: Jürgen Köcher, Langenfeld (DE); Christian Wamprecht, Neuss (DE)

(73) Assignee: Bayer Materialscience AG, Leverkusen (DE)

(21) Appl. No.: 13/496,576

(22) PCT Filed: Sep. 4, 2010
(86) PCT No.: PCT/EP10/05440

§ 371 Date: Mar. 16, 2012

(30) Foreign Application Priority Data
Sep. 17, 2009 (EP) 09011851.4

(51) Int. Cl. C08K 5/3415; C08L 75/04; C08K 5/20; A61K 47/34; C08K 5/06; C08K 5/07; C08K 5/05; C09D 175/04; C08G 18/28; C08K 5/21

(52) U.S. Cl. 514/772.3; 528/85; 524/590; 524/233; 524/211; 524/104; 524/366; 524/356; 524/379

(57) ABSTRACT

The invention relates to novel polyurethane ureas which can be used in the production of hydrophilic coatings on various substrates.

The present invention relates to innovative polyurethaneureas which can be used for producing hydrophilic coatings on a very wide variety of substrates.

Particularly in the medical sector, hydrophilic coatings on surfaces of medical devices are important since their use can be greatly improved as a result. The insertion and displacement of urinary or blood-vessel catheters is made easier by the fact that hydrophilic surfaces in contact with blood or urine adsorb a film of water. This reduces the friction between the catheter surface and the vessel walls, and so the catheter is easier to insert and move. Direct watering of the devices prior to the intervention can also be performed in order to reduce friction through the formation of a homogeneous water film. The patients concerned experience less pain and the risk of injuries to the vessel walls is reduced by such measures. Furthermore, when catheters are being used, there is always the risk of formation of blood clots. In this context, hydrophilic coatings are generally considered to be useful for antithrombogenic coatings.

Hydrophilic coatings for medical devices are required to have a high mechanical robustness. This is necessary in particular since their use is accompanied by mechanical forces which in some cases are considerable. This is true, for example, of catheters and stents when they are introduced into vessels. From standpoints of physiology alone, any delamination of the coating must absolutely be avoided. In addition, however, the coating must also be flexible enough to allow, for instance, the compression of a stent or catheter.

U.S. Pat. No. 5,589,563 discloses coatings with surface-modified end groups that can be used inter alia for the coating of medical devices. These coatings, however, still do not have satisfactory physical properties. On the one hand, for instance, their hydrophilicity is not sufficiently high. Lastly, the coatings are generally not sufficiently stably formed, i.e. their mechanical robustness is too low.

European application No. 08153055.2, unpublished at the priority date of the present specification, discloses hydrophilic coatings comprising polyurethaneureas which are based on a specific combination of polycarbonate polyols as synthesis components and copolymers of ethylene oxide and propylene oxide as end groups. In terms of their mechanical properties, these coatings as well still do not satisfy all of the requirements.

It was an object of the invention, therefore, to provide a base coating material which can be used for producing hydrophilic coatings of high mechanical robustness more particularly on medical devices such as stents or catheters.

This object is achieved by means of a polyurethaneurea terminated with at least one copolymer unit of polyethylene oxide and polypropylene oxide, which has at least one structural unit of the formula (I)

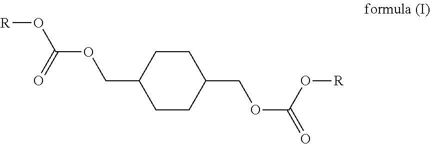

which is linked to the polymer chain by at least one bond R.

The coatings obtainable using the polyurethaneureas of the invention are notable for a high hydrophilicity and a high mechanical robustness.

Polyurethaneureas for the purposes of the present invention are polymeric compounds which have

(a) at least two repeating units containing urethane groups, of the following general structure

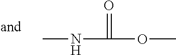

(b) at least one repeating unit containing urea groups:

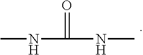

The polyurethaneureas according to the invention preferably have no ionic or ionogenic modification. By this is meant, in the context of the present invention, that they have substantially no ionic groups, such as, in particular, sulphonate, carboxylate, phosphate and phosphonate groups.

The term “substantially no ionic groups” means, in the context of the present invention, that the resulting coating of the polyurethaneurea has ionic groups with a fraction of in general not more than 2.50% by weight, in particular not more than 2.00% by weight, preferably not more than 1.50% by weight, more preferably not more than 1.00% by weight, especially not more than 0.50% by weight, more especially no ionic groups. Hence it is preferred in particular that the polyurethaneurea has no ionic groups, since high concentrations of ions in organic solution mean that the polymer is no longer sufficiently soluble and hence that no stable solutions can be obtained. If the polyurethaneurea does have ionic groups, the groups in question are preferably carboxylates and sulphonates.

The polyurethaneureas are preferably substantially linear molecules, but may also be branched, although this is less preferred. Substantially linear molecules in the context of the present invention are systems with low levels of incipient crosslinking, the parent polycarbonate polyol component preferably having an average hydroxy functionality of preferably 1.7 to 2.3, more preferably 1.8 to 2.2, very preferably 1.9 to 2.1.

The number-average molecular weight of the polyurethaneureas is preferably 1000 to 200, 000 g/mol, more preferably from 3000 to 100, 000 g/mol. This number-average molecular weight is measured against polystyrene as standard in dimethylacetamide at 30° C.

The polyurethaneureas may be prepared by reacting synthesis components which comprise at least one polycarbonate polyol component a), at least one polyisocyanate component b), at least one polyoxyalkylene ether component c), at least one diamine and/or amino alcohol component d) and optionally a further polyol component e).

The invention therefore likewise provides a process for preparing the polyurethaneureas according to the invention, in which a polycarbonate polyol component a), at least one polyisocyanate component b), at least one polyoxyalkylene ether component c), at least one diamine and/or amino alcohol component d) and, if desired, a further polyol component are reacted with one another.

Component a) comprises at least one polycarbonate polyol a1), which is obtained by reacting carbonic acid derivatives such as diphenyl carbonate, dimethyl carbonate or phosgene with difunctional alcohols of the formula (II)

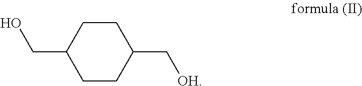

For the preparation in a pressure reactor and at elevated temperature, 1,4-cyclohexanedimethanol is reacted with diphenyl carbonate, dimethyl carbonate or phosgene. Reaction with dimethyl carbonate is preferred. Where dimethyl carbonate is used, the methanol elimination product is removed by distillation in a mixture with excess dimethyl carbonate.

The preparation of the polycarbonate polyols by means of dimethyl carbonate takes place at temperatures of up to 240° C., preferably up to 220° C. and more preferably up to 200° C. It is possible to operate either at atmospheric pressure or else under a pressure of up to 15 bar, preferably up to 10 bar and more preferably up to 5 bar. Moreover, the preparation of the polycarbonates in certain substeps (dewatering, distillation) also takes place under a reduced pressure of <500 mbar, preferably <100 mbar and more preferably <20 mbar.

The polycarbonate polyols a1) based on diols of the formula (II) have molecular weights, determined through the OH number, of preferably 200 to 10,000 g/mol, more preferably 300 to 8000 g/mol and very preferably 400 to 6000 g/mol.

Component a) is preferably a mixture of the aforementioned polycarbonate polyols a1) based on diols of the formula (II) and further polycarbonate polyols a2).

Such further polycarbonate polyols a2) preferably have average hydroxyl functionalities of 1.7 to 2.3, more preferably of 1.8 to 2.2, very preferably of 1.9 to 2.1.

Furthermore, the polycarbonate polyols a2) have molecular weights, determined through the OH number, of preferably, 400 to 6000 g/mol, more preferably from 500 to 5000 g/mol, in particular of 600 to 3000 g/mol, which are obtainable, for example, by reaction of carbonic acid derivatives, such as diphenyl carbonate, dimethyl carbonate or phosgene, with polyols, preferably diols. Suitable such diols include, for example, ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The polycarbonate polyols a2) contain preferably 40% to 100% by weight of hexanediol, preferably 1,6-hexanediol and/or hexanediol derivatives, preferably those which as well as terminal OH groups have ether groups or ester groups, examples being products obtained by reacting 1 mol of hexanediol with at least 1 mol, preferably 1 to 2 mol of caprolactone or by etherifying hexanediol with itself to give the dihexylene or trihexylene glycol, as synthesis components. Polyether-polycarbonate diols can be used as well. The hydroxyl polycarbonates ought to be substantially linear. Where appropriate, however, they may be slightly branched as a result of the incorporation of polyfunctional components, especially low molecular weight polyols. Examples of polyols suitable for this purpose include glycerol, trimethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preference is given to such polycarbonates a2) based on hexane-1,6-diol and also on modifying co-diols such as for example, butane-1,4-diol or else on ε-caprolactone. Further preferred polycarbonate diols a2) are those based on mixtures of hexane-1,6-diol and butane-1,4-diol.

In one preferred embodiment, a mixture is used in a) of the polycarbonate polyols a1) and those polycarbonate polyols a2) based on hexane-1,6-diol, butane-1,4-diol or mixtures thereof.

In the case of mixtures of the constituents a1) and a2), the fraction of a1) as a proportion of the mixture is preferably at least 5 mol %, more preferably at least 10 mol %, based on the total molar amount of polycarbonate.

The polyurethaneureas additionally have units which derive from at least one polyisocyanate as synthesis component b).

As polyisocyanate b) it is possible to use all of the aromatic, araliphatic, aliphatic and cycloaliphatic isocyanates that are known to the person skilled in the art and have an average NCO functionality ≧1, preferably ≧2, individually or in any desired mixtures with one another, it being immaterial whether they have been prepared by phosgene processes or phosgene-free processes. They may also have iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, having a carbon skeleton (without the NCO groups present) of 3 to 30, preferably 4 to 20 carbon atoms.

Particularly preferred compounds of component b) correspond to the type mentioned above with aliphatically and/or cycloaliphatically attached NCO groups, such as, for example, bis(isocyanatoalkyl)ethers, bis- and tris(isocyanatoalkyl)benzenes, -toluenes, and -xylenes, propane diisocyanates, butane diisocyanates, pentane diisocyanates, hexane diisocyanates (e.g., hexamethylene diisocyanate, HDI), heptane diisocyanates, octane diisocyanates, nonane diisocyanates (e.g. trimethyl-HDI (TMDI) generally in the form of a mixture of the 2,4,4- and 2,2,4-isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(isocyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component b) are hexamethylene diisocyanate (HDI), Trimethyl-HDI (TMDI), 2-methylpentane-1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate (IMCI) and/or 4,4′-bis(isocyanato-cyclohexyl)methane (H₁₂MDI) or mixtures of these isocyanates. Further examples are derivatives of the aforementioned diisocyanates with uretdione, isocyanurate, urethane, allophanate, biuret, iminooxadiazinedione and/or oxadiazinetrione structure and with more than two NCO groups.

The amount of the constituent b) in the preparation of the polyurethaneureas is preferably 1.0 to 3.5 mol, more preferably 1.0 to 3.3 mol, in particular 1.0 to 3.0 mol, based in each case on the amount of the compounds of component a).

The polyurethaneureas have units which derive from a copolymer of polyethylene oxide and polypropylene oxide as synthesis component c). These copolymer units are present in the form of end groups in the polyurethaneurea and have the effect of a particularly advantageous hydrophilicization.

Nonionically hydrophilicizing compounds c) of this kind are, for example, monofunctional polyalkylene oxide polyether alcohols that have on average 5 to 70, preferably 7 to 55, ethylene oxide units per molecule, of the kind obtainable in a manner known per se by alkoxylating suitable starter molecules (e.g. in Ullmanns Enzyklopädie der technischen Chemie, 4th edition, Volume 19, Verlag Chemie, Weinheim, pp. 31-38).

Suitable starter molecules are, for example, saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine and also heterocyclic secondary amines such as morpholine, pyrrolidine, piperidine or 1H-pyrazole. Preferred starter molecules are saturated monoalcohols. Particular preference is given to using diethylene glycol monobutyl ether as starter molecule.

The alkylene oxides, ethylene oxide and propylene oxide, can be used in any order or else in a mixture in the alkoxylation reaction.

The polyalkylene oxide polyether alcohols are mixed polyalkylene oxide polyethers of ethylene oxide and propylene oxide, and preferably at least 30 mol %, more preferably at least 40 mol % of their alkylene oxide units are composed of ethylene oxide units. Preferred nonionic compounds are monofunctional mixed polyalkylene oxide polyethers, which have at least 40 mol % of ethylene oxide units and not more than 60 mol % of propylene oxide units based on the total fraction of alkylene oxide units.

The number-average molar weight of the polyoxyalkylene ether is preferably 500 g/mol to 5000 g/mol, more preferably 1000 g/mol to 4000 g/mol, in particular 1000 to 3000 g/mol.

The amount of constituent c) in the preparation of the polyurethaneureas is preferably 0.01 to 0.5 mol, more preferably 0.02 to 0.4 mol, in particular 0.04 to 0.3 mol, based in each case on the amount of the compounds of component a).

It has been possible to show that the polyurethaneureas with end groups which are based on mixed polyoxyalkylene ethers of polyethylene oxide and polypropylene oxide are particularly suitable for producing coatings having a high hydrophilicity.

The polyurethaneureas may have units which derive from at least one'diamine or amino alcohol as a synthesis component, and serve as what are known as chain extenders d).

Such chain extenders are, for example diamines or polyamines and also hydrazides, examples being hydrazine, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, tetramethyl-1,3- and -1,4-xylylenediamine and 4,4′-diaminodicyclohexylmethane, dimethylethylenediamine, hydrazine, adipic dihydrazide, 1,4-bis(aminomethyl)cyclohexane, 4,4′-diamino-3,3′-dimethyldicyclohexylmethane and other (C₁-C₄)-di- and tetraalkyl-dicyclohexylmethanes, e.g. 4,4′-diamino-3,5-diethyl-3′,5′-diisopropyldicyclohexylmethane.

Suitable diamines or amino alcohols are generally diamines or amino alcohols of low molecular weight which contain active hydrogen whose reactivity towards NCO groups differs, such as compounds which as well as primary amino groups also have secondary amino groups, or as well as an amino group (primary or secondary) also have OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and with particular preference diethanolamine.

Constituent d) of the polyurethaneureas can be used as a chain extender in their preparation.

The amount of constituent d) in preparing the polyurethaneureas is preferably 0.1 to 1.5 mol, more preferably 0.2 to 1.3 mol, in particular 0.3 to 1.2 mol, based in each case on the amount of the compounds of component a).

In a further embodiment, the polyurethaneureas comprise additional units which derive from at least one further polyol as a synthesis component.

The further, low molecular weight polyols e) that are used to synthesize the polyurethaneureas generally have the effect of stiffening and/or of branching of the polymer chain. The molecular weight is preferably 62 to 500 g/mol, more preferably 62 to 400 g/mol, in particular 62 to 200 g/mol.

Suitable polyols may contain aliphatic, alicyclic or aromatic groups. Mention may be made here, for example, of the low molecular weight polyols having up to about 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), and also trimethylolpropane, glycerol or pentaerythritol and mixtures thereof and also, where appropriate of further low molecular weight polyols. Ester diols can be used as well, such as, for example, α-hydroxybutyl-ε-hydroxycaproic esters, co-hydroxyhexyl-γ-hydroxybutyric esters, adipic acid β-hydroxyethyl ester or terephthalic acid bis(β-hydroxyethyl) ester.

The amount of constituent e) in preparing the polyurethaneureas is preferably 0.05 to 1.0 mol, more preferably 0.05 to 0.5 mol, in particular 0.1 to 0.5 mol, based in each case on the amount of the compounds of component a).

The reaction of the isocyanate-containing component b) with the hydroxy- or amine-functional compounds a), c), d) and where appropriate, e), is typically accomplished while observing a slight NCO excess over the reactive hydroxy or amine compounds. At the end point of the reaction, as a result of the attainment of a target viscosity, there always are residues of active isocyanate remaining. These residues must, in the case of a solvent polymerization, be blocked so that there is no reaction with large polymer chains. Such a reaction leads to the three-dimensional crosslinking and gelling of the batch. A polyurethaneurea solution of that kind can no longer be processed. Typically the batches contain high quantities of alcohols. These alcohols block the remaining isocyanate groups within a number of hours of standing or stirring of the batch at room temperature. In the case of the preparation of an aqueous polyurethane dispersion, the excess 0.25 isocyanate groups are customarily hydrolysed by the water that is added. Alternatively, the excess isocyanate groups can also be blocked by deliberate addition of a component f) before the addition of the water or before the alcoholysis by the solvent alcohols.

If the residual isocyanate content has been blocked during the preparation of the polyurethaneureas in an organic solution or in an aqueous dispersion, they also have, as synthesis components, monomers f) which are in each case located at the chain ends and cap them.

These synthesis components derive on the one hand from monofunctional compounds that are reactive with NCO groups, such as monoamines, especially mono-secondary amines, or monoalcohols. Examples that may be mentioned here include ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxy-propylamine, dimethylamine, diethylamine, dipropylamine, dibutylamine, N-methylamino-propylamine, diethyl(methyl)aminopropylamine, morpholine, piperidine and suitable substituted derivatives thereof.

Since the building blocks f) are used essentially in order to destroy the NCO excess, the amount required is dependent essentially on the amount of the NCO excess, and cannot be specified in general terms.

Preferably these building blocks are omitted during the synthesis. Here, in the case of the organic polyurethaneurea solutions, unreacted isocyanate is preferably converted to terminal urethanes by solvent alcohols that are present at very high concentrations. If an aqueous dispersion is prepared, the excess isocyanate groups are usually hydrolysed by the dispersion water that has been added.

For preparing the polyurethaneureas in organic solution, the polycarbonate polyol component a), the polyisocyanate, the monofunctional polyether alcohol and, where appropriate, the polyol, are reacted with one another in the melt or in solution until all of the hydroxyl groups have been consumed.

The stoichiometry used in this case between the individual synthesis components participating in the reaction is a product of the proportions mentioned above.

The reaction takes place at a temperature of preferably 60 to 110° C., more preferably of 75 to 110° C., in particular of 90 to 110° C., with preference being given to temperatures around 110° C. on account of the rate of the reaction. Higher temperatures can likewise be employed, but then, in certain cases, and dependant on the individual constituents used, there is a risk of decomposition events and instances of discoloration occurring in the resultant polymer.

In the case of the prepolymer of isocyanate and all of the components having hydroxyl groups, reaction in the melt is preferred, albeit with a risk of excessive viscosities on the part of the fully reacted mixtures. In such cases it is also advisable to add solvents. However, there ought as far as possible to be not more than approximately 50% by weight of solvents present, since otherwise the dilution significantly retards the reaction rate.

In the case of the reaction of isocyanate and the components having hydroxyl groups, the reaction can take place in the melt in a period of 1 hour to 24 hours. Small additions of solvent lead to a retardation, but the reaction times are within the same time periods.

The sequence of the addition and/or reaction of the individual constituents may deviate from the sequence indicated above. This may be of advantage in particular when the mechanical properties of the resultant coatings are to be modified. Where, for example, all of the components having hydroxyl groups are reacted simultaneously, a mixture of hard segments and soft segments is produced. Where, for example, the low molecular weight polyol is added after the polycarbonate polyol component, defined blocks are obtained, which may result in different properties in the resulting coatings. The present invention is therefore not confined to a particular sequence of the addition and/or reaction of the individual constituents of the polyurethane coating.

If a polyurethaneurea is prepared in an organic solution, the further solvent is then added and the chain extender diamine and/or the dissolved chain extender amino alcohol (synthesis component (d)), in solution where appropriate, is or are added.

The further addition of the solvent takes place preferably in steps, in order not to retard the reaction unnecessarily, as would happen if the entire amount of solvent were to be added, for example, at the start of the reaction. Furthermore, a high solvent content at the beginning of the reaction dictates a relatively low temperature, which is at least co-determined by the nature of the solvent. This leads as well to the retardation of the reaction.

When the target viscosity has been obtained, the remaining residues of NCO can be blocked by a monofunctional aliphatic amine. The isocyanate groups that remain are preferably blocked by reaction with the alcohols that are present in the solvent mixture.

Suitable solvents for the preparation and application of the polyurethaneurea solutions of the invention include all conceivable solvents and solvent mixtures such as dimethylformamide, N-methylacetamide, tetramethylurea, N-methylpyrrolidone, aromatic solvents such as toluene, linear and cyclic esters, ethers, ketones and alcohols. Examples of esters and ketones are, for example, ethyl acetate, butyl acetate, acetone, γ-butyrolactone, methyl ethyl ketone and methyl isobutyl ketone.

Preference is given to mixtures of alcohols with toluene. Examples of the alcohols which are used together with the toluene are ethanol, n-propanol, isopropanol and 1-methoxy-2-propanol.

In general the amount of solvent used in the reaction is such as to give approximately 10% to 50% strength by weight solutions, preferably approximately 15% to 45% strength by weight solutions and more preferably approximately 20% to 40% strength solutions.

The solids content of the polyurethane solutions is generally in the range from 5 to 60% by weight, preferably from 10 to 40% by weight. For coating experiments, the polyurethane solutions can be diluted arbitrarily with toluene/alcohol mixtures in order to allow variable adjustment of the thickness of the coating. All concentrations from 1% to 60% by weight are possible; concentrations in the 1% to 40% by weight range are preferred. In this context it is possible to achieve any desired coat thicknesses, such as for example from a few hundred nm up to a few 100 μm, with higher and lower thicknesses also being possible in the context of the present invention.

The polyurethaneurea dispersions of the invention are prepared preferably, by the process known as the acetone process. For preparing the polyurethaneurea dispersion by this acetone process, customarily, the constituents a), c) and e), which must not contain any primary or secondary amino groups, and the polyisocyanate component b), for the preparation of an isocyanate-functional polyurethane prepolymer, are introduced in whole or in part, are optionally diluted with a solvent which is miscible with water but inert towards isocyanate groups, and heated to temperatures in the range from 50 to 120° C. The isocyanate addition reaction can be accelerated using the catalysts that are known in polyurethane chemistry, an example being dibutyltin dilaurate. Synthesis without catalyst is preferred.

Suitable solvents are the customary aliphatic, keto-functional solvents such as, for example, acetone, butanone, which may be added not only at the beginning of the preparation but also optionally in portions later on as well. Acetone and butanone are preferred. Other solvents such as, for example, xylene, toluene, cyclohexane, butyl acetate, methoxypropyl acetate and solvents with ether units or ester units may likewise be used and may be wholly or partly removed by distillation or may remain entirely in the dispersion.

Then any constituents of c) and e) not yet added at the beginning of the reaction are metered in.

In a preferred way, the prepolymer is prepared without addition of solvent, and is diluted with a suitable solvent, preferably acetone, only for the chain extension.

The conversion to the prepolymer takes place partly or completely, but preferably completely. In this way, polyurethane prepolymers containing free isocyanate groups are obtained, in bulk or in solution.

Subsequently, in a further process step, if it has not yet already taken place or has taken place only partially, the resulting prepolymer is dissolved using aliphatic ketones such as acetone or butanone.

Subsequently, possible NH₂—, NH-functional and/or OH-functional components are reacted with the remaining isocyanate groups. This chain extension/chain termination may be carried out either in solvent prior to dispersing, during dispersing, or in water after dispersing. The chain extension is carried out preferably before the dispersing in water.

Where compounds meeting the definition of d) with NH₂ or NH groups are used for the chain extension, the chain extension of the prepolymers takes place preferably prior to the dispersing.

The degree of chain extension, in other words, the equivalents ratio of NCO-reactive groups of the compounds used for chain extension to free NCO groups of the prepolymer, is preferably between 40% to 150%, more preferably between 50% to 120%, more particularly between 60% to 120%.

The aminic components d) may optionally be used in water-diluted or solvent-diluted form in the process of the invention, individually or in mixtures, and in principle any sequence of the addition is possible.

If the remaining residue isocyanate groups are not to be hydrolysed by the dispersion water, it is possible here to add a sufficient amount of component f) to block the isocyanate groups before the dispersion water is added.

If water or organic solvents are used as diluents, then the diluent content is preferably 70% to 95% by weight.

The preparation of the polyurethane dispersion from the prepolymers takes place following the chain extension. It is effected either by introducing the dissolved and chain-extended polyurethane polymer into the dispersion water, optionally with strong shearing, such as vigorous stirring, for example, or, conversely, the dispersion water is stirred into the prepolymer solutions. Preferably, the water is added to the dissolved prepolymer.

The solvent still present in the dispersions after the dispersing step is typically removed subsequently by distillation. Removal actually during the dispersion is likewise possible.

The solids content of the polyurethane dispersion after the synthesis is in the range from 20% to 70% by, weight, preferably 20% to 65% by weight. For coating tests, these dispersions can be diluted arbitrarily with water, in order to allow variable adjustment of the thickness of the coating. All concentrations from 1% to 60% by weight are possible, preference being given to concentrations in the 1% to 40% by weight range.

The polyurethaneureas may further comprise additives and constituents that are customary for the particular end use.

One example of such are pharmacological actives, medicaments and additives, which promote the release of pharmacological actives (“drug-eluting additives”).

Pharmacological actives and medicaments which can be used in the coatings of the invention on the medical devices are, for example, thromboresistant agents, antibiotic agents, anti-tumour agents, growth hormones, antiviral agents, antiangiogenic agents, angiogenic agents, antimitotic agents, anti-inflammatory agents, cell cycle regulators, genetic agents, hormones and also their homologues, derivatives, fragments, pharmaceutical salts and combinations thereof.

Specific examples of such pharmacological actives and medicaments hence include thromboresistant (non-thrombogenic) agents and other agents for suppressing an acute thrombosis, stenosis or late re-stenosis of the arteries, examples being heparin, streptokinase, urokinase, tissue plasminogen activator, anti-thromboxan-B₂ agent; anti-B-thromoboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc.

A growth factor likewise may be utilized as a medicament in order to suppress subintimal fibromuscular hyperplasia of the arterial stenosis site, or any other cell growth inhibitor can be utilized at the stenosis site.

The pharmacological active or medicament may also be composed of a vasodilator, in order to counteract vasospasm. One example in this case is an antispasm agent such as papaverine. The medicament may be a vasoactive agent such as calcium antagonists, per se, or α- and β-adrenergic agonists or antagonists. In addition the therapeutic agent may be a biological adhesive such as cyanoacrylate in medical grade or fibrin, which is used, for example, for bonding a tissue valve to the wall of a coronary artery.

The therapeutic agent may further be an antineoplastic agent such as 5-fluorouracil, preferably with a controlling releasing vehicle for the agent, (for example, for the use of an ongoing controlled releasing antineoplastic agent at a tumour site).

The therapeutic agent may be an antibiotic, preferably in combination with a controlling releasing vehicle for ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include the following:
(a) heparin, heparin sulphate, hirudin, hyaluronic acid, chondroitin sulphate, dermatan sulphate, keratin sulphate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;
(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkoids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof;
   (d) antiviral agents such as amantadine, rimantadine, rabavirin, idoxuridine, vidarabin, trifluridine, acyclovir, ganciclorir, zidovudine, phosphonoformates, interferons, their homologues, analogues, fragments, derivatives, pharmaceutical salts and mixtures thereof; and
   e) antiflammatory agents such as, for example, ibuprofen, dexamethasone or methylprednisolone.

To generate surfaces having infestation-inhibiting properties, the coating compositions of the invention may comprise the active infestation inhibitors known from the prior art. Their presence generally boosts the already outstanding infestation-inhibiting properties of the surfaces produced with the coating compositions themselves.

Further additions such as, for example, antioxidants or pigments may likewise be used. Additionally it is possible, where appropriate, to use further additions as well, such as hand agents, dyes, matting agents, UV stabilizers, light stabilizers, hydrophobicizers and/or flow control assistants.

The polyurethaneureas of the invention can be used to form a coating for example on a medical device.

The term “medical device” is to be understood broadly in the context of the present invention. Suitable, non-limiting examples of medical devices (including instruments) are contact lenses; cannulas; catheters, for example urological catheters such as urinary catheters or ureteral catheters; central venous catheters; venous catheters or inlet or outlet catheters; dilation balloons; catheters for angioplasty and biopsy; catheters used for introducing a stent, an embolism filter or a vena caval filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide rods, insertion guides and the like; vascular plugs; pacemaker components; cochlear implants; dental implant tubes for feeding, drainage tubes; and guide wires;

The polyurethaneureas according to the invention may be used, furthermore, for producing protective coatings, for example for gloves, stents and other implants; external (extracorporeal) blood lines (blood-carrying tubes); membranes; for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical devices are selected from catheters, endoscopes, laryngoscopes, endotracheal tubes, feeding tubes, guide rods, stents, and other implants.

In addition to the hydrophilic properties of improving the lubricity, the coating compositions provided in accordance with the invention are also notable for a high level of blood compatibility. This makes working with these coatings advantageous in blood contact in particular. The materials exhibit reduced coagulation tendency in blood contact as compared with polymers of the prior art.

Systems which release active substances and are based on the hydrophilic coating materials of the invention are also conceivable outside medical technology, as for example for applications in crop protection as a carrier material for actives. The entire coating may in that case be considered an active-releasing system and may be used, for example, to coat seed (seed grains). As a result of the hydrophilic properties of the coating, the active it contains is able to emerge in the moist earth and develop its intended effect, without adversely affecting the capacity of the seed to germinate. In the dry state, however, the coating composition binds the active securely to the seed, and so, for example, the active is not detached, when the seed grain is being fired into the soil by the broadcasting machine; as a result of such detachment, the active could develop unwanted effects, for example, on the fauna that are present (jeopardizing bees by insecticides intended per se to prevent the attack of insects on the seed grain in the soil).

Beyond their application as a coating for medical devices, the polyurethane solutions according to the invention can also be utilized for further technical applications in the non-medical sector.

Thus, the polyurethaneureas according to the invention serve for producing coatings as protection of surfaces against fogging with moisture, for the production of easy-to-clean or self-cleaning surfaces. These hydrophilic coatings also reduce the pick-up of dirt and prevent the formation of water spots. Conceivable applications in the exterior sector are, for example, windows and roof lights, glass facades or Plexiglas roofs. In the interior sector, materials of this kind can be utilized for the coating of surfaces of sanitary equipment. Further applications are the coating of spectacle lenses or of packaging materials such as food packaging for the purpose of preventing moisture fogging or droplet formation due to condensed water.

The polyurethaneureas according to the invention are also suitable for treating surfaces in contact with water for the purpose of reducing infestation. This effect is also referred to as the antifouling effect. One very important application of this antifouling effect is in the area of the underwater coatings on ships' hulls. Ships' hulls without an antifouling treatment very quickly become infested by marine organisms, leading to increased friction and hence to a reduction in the possible speed and a higher consumption of fuel. The coating materials of the invention reduce or prevent infestation by marine organisms, and prevent the above-described disadvantages of this infestation. Further applications in the area of antifouling coatings are articles for fishing such as fishing-nets and also all metallic substrates in underwater use, such as pipelines, offshore drilling platforms, locks and lock gates, etc. Hulls which have surfaces generated with the coating materials of the invention, especially below the water line, also possess a reduced frictional resistance, and so ships thus equipped either have a reduced fuel consumption or achieve higher speeds. This is of interest in particular in the sporting boat sector and in yacht building.

A further important field for application of the abovementioned hydrophilic coating materials is the printing industry. By means of the coatings of the invention, hydrophobic surfaces can be made hydrophilic and as a result can be printed with polar printing inks, or can be printed using ink-jet technology.

A further field for application of the hydrophilic coatings of the invention is in formulations for cosmetic applications.

Coatings of the polyurethaneureas according to the invention can be applied by means of a variety of methods. Examples of suitable coating techniques for these solutions include knife coating, printing, transfer coating, spraying, spin coating or dipping.

A wide variety of substrates can be coated, such as metals, textiles, ceramics and plastics. Preference is given to coating medical devices manufactured from plastic or metal. Examples of metals that can be mentioned include the following: medical stainless steel and nickel titanium alloys. Many polymer materials are conceivable from which the medical devices may be constructed, examples being polyamide; polystyrene; polycarbonate; polyethers; polyesters; polyvinyl acetate; natural and synthetic rubbers; block copolymers of styrene and unsaturated compounds such as ethylene, butylene and isoprene; polyethylene or copolymers of polyethylene and polypropylene; silicone; polyvinyl chloride (PVC) and polyurethanes. For better adhesion of the hydrophilic polyurethaneureas to the medical device, further suitable coatings may be applied as a base before these hydrophilic coating materials are applied.

### EXAMPLES

The NCO content of the resins described in the inventive and comparative examples was determined by titration in accordance with DIN EN ISO 11909.

The solids contents were determined in accordance with DIN-EN ISO 3251. Polyurethaneurea dispersion or solution (1 g) was dried at 115° C. at a constant weight (15-20 min) using an infrared drier.

The average particle sizes of the polyurethaneurea dispersions or solution were measured using the High Performance Particle Sizer (HPPS 3.3) from Malvern Instruments.

Unless noted otherwise, the amounts reported in % are to be understood as % by weight and relate to the dispersion or solution obtained.

The tensile strengths were determined in accordance with DIN 53504.

Viscosity measurements were carried out using the Physics MCR 51 Rheometer from Anton Paar GmbH, Ostfildern, Germany.

### Substances Used and Abbreviations:

Desmophen® C2200: polycarbonate polyol, OH number 56 mg KOH/g, number-average molecular weight 2000 g/mol (Bayer MaterialScience Ag, Leverkusen, De)
Desmophen® C 1200: polycarbonate polyol, OH number 56 mg KOH/g, number-average molecular weight 2000 g/mol (Bayer MaterialScience Ag, Leverkusen, De)
Polyether LB 25: monofunctional polyether based on ethylene oxide/propylene oxide, number-average molecular weight 2250 g/mol, OH number 25 mg KOH/g (Bayer MaterialScience Ag, Leverkusen, De)
1,4-Cyclohexanedimethanol product from Eastman, Kingsport, Tenn., USA

### Polycarbonate Diols

### Example 1

### Preparation of a Cycloaliphatic Polycarbonate Diol Based on Cyclohexanedimethanol with a Number-Average Molecular Weight of Approximately 1000 g/mol

A 16 l pressure reactor with top-mounted distillation attachment, stirrer and receiver was charged with 3042.4 g of cyclohexanedimethanol under a nitrogen atmosphere, and this initial charge was dewatered for 2 hours at 90° C. under a reduced pressure of 20 mbar. It was then blanketed with nitrogen and a reflux condenser was installed. Subsequently, 0.7 g of yttrium(III) acetylacetonate and 2377.9 g of dimethyl carbonate were added at 90° C. Under a nitrogen atmosphere, the reaction mixture was heated to 135° C. over 2 hours and held under reflux with stirring for 24 hours. Thereafter the temperature was raised to 150° C. and the batch was stirred at this temperature for 4 hours. The temperature was then raised to 180° C. and the batch was stirred at this temperature for a further 4 hours. It was then cooled to 130° C. and the reaction vessel was converted for the subsequent distillation. Then the elimination product, methanol, was removed in a mixture with dimethyl carbonate by distillation, the temperature being raised in steps to 180° C. This was followed by at 180° C. for 6 hours under a reduced pressure of 20 mbar. In the course of this operation, methanol was removed further from the reaction mixture, in a mixture with dimethyl carbonate.

After air had been admitted and the reaction mixture cooled to room temperature, a yellowish solid polycarbonate diol was obtained that had the following characteristics:

Mₙ=984 g/mol; OH number=114 mg KOH/g.

### Example 2

### Preparation of a Cycloaliphatic Polycarbonate Diol Based on Cyclohexanedimethanol with a Number-Average Molecular Weight of Approximately 500 g/mol

Procedure as in Example 1, using 3119.5 g of cyclohexanedimethanol, 0.7 g of yttrium(III) acetylacetonate and 1977.3 g of dimethyl carbonate.

This gave a yellowish solid polycarbonate diol that had the following characteristics:

Mₙ=524 g/mol; OH number=214 mg KOH/g.

### Solutions of Polyurethaneurea

### Example 3a (Comparative)

195.4 g of Desmophen C 2200, 30.0 g of LB 25 and 47.8 g of 4,4′-bis(isocyanato-cyclohexyl)methane (H₁₂MDI) were reacted at 110° C. to a constant NCO content of 2.4%. The mixture was allowed to cool and was diluted with 350.0 g of toluene and 200 g of isopropanol. At room temperature, a solution of 11.8 g of isophoronediamine in 94.0 g of 1-methoxypropan-2-ol was added. After the end of the increase in molar weight, and the attainment of the desired viscosity range, stirring was continued for 5 hours at room temperature in order to block the residual isocyanate content with isopropanol. This gave 929 g of a 31.9% strength solution of polyurethaneurea in toluene/isopropanol/1-methoxypropan-2-ol with a viscosity of 37,100 mPas at 22° C.

### Example 4a (Inventive)

97.8 g of Desmophen C 2200, 48.9 g of polycarbonate diol of Example 1, 30.0 g of LB 25 and 47.8 g of 4,4′-bis(isocyanatocyclohexyl)methane (H₁₂MDI) were reacted at 110° C. to a constant NCO content of 2.8%. The mixture was allowed to cool and was diluted with 320.0 g of toluene and 185 g of isopropanol. At room temperature, a solution of 12.3 g of isophoronediamine in 100 g of 1-methoxypropan-2-ol was added. After the end of the increase in molar weight, and the attainment of the desired viscosity range, stirring was continued for 16 hours at room temperature in order to block the residual isocyanate content with isopropanol. This gave 841.8 g of a 28.6% strength solution of polyurethaneurea in toluene/isopropanol/1-methoxypropan-2-ol with a viscosity of 14,100 mPas at 22° C.

### Example 5a (Inventive)

146.8 g of Desmophen C 2200, 24.5 g of polycarbonate diol of Example 1, 30.0 g of LB 25 and 47.8 g of 4,4′-bis(isocyanatocyclohexyl)methane (H₁₂MDI) were reacted at 110° C. to a constant NCO content of 2.6%. The mixture was allowed to cool and was diluted with 320.0 g of toluene and 185 g of isopropanol. At room temperature, a solution of 11.4 g of isophoronediamine in 90 g of 1-methoxypropan-2-ol was added. After the end of the increase in molar weight, and the attainment of the desired viscosity range, stirring was continued for 22 hours at room temperature in order to block the residual isocyanate content with isopropanol. This gave 855.5 g of a 30.9% strength solution of polyurethaneurea in toluene/isopropanol/1-methoxypropan-2-ol with a viscosity of 24,400 mPas at 22° C.

### Example 6a

### Contact Angles and 100% Moduli of Comparative Example 3a Against Inventive Examples 4a and 5a

### 1. Production of the Coatings for the Measurement of the Static Contact Angle

The coatings for the measurement of the static contact angle were produced on glass slides measuring 25×75 mm using a spin coater (RC5 Gyrset 5, Karl Süss, Garching, Germany). For this purpose, a slide was clamped on to the sample plate of the spin coater and covered homogeneously with about 2.5-3 g of organic 15% strength polyurethane solution. All of the organic polyurethane solutions were diluted to a polymer content of 15% using a solvent mixture of 65% by weight toluene and 35% by weight isopropanol. Rotation of the sample plate at 1300 revolutions per minute for 20 seconds gave a homogeneous coating, which was dried at 100° C. for 1 h and then at 50° C. for 24 h. The coated slides obtained were subjected directly to a contact angle measurement.

A static contact angle measurement was performed on the resulting coatings on the slides. Using the video contact angle measuring instrument OCA20 from Dataphysics, with computer-controlled injection, 10 drops of Millipore water were applied to the specimen, and their static wetting contact angle was measured. Beforehand, using an antistatic drier, the static charge (if present) on the sample surface was removed.

### 2. Production of the Coatings for the Measurement of the 100% Modulus

Films are produced on release paper using a 200 μm coating bar, and are dried at 100° C. for 15 minutes. Punched shapes are investigated in accordance with DIN 53504.

### 3. Results of Investigation

TABLE 1
Contact angles and 100% moduli of the
films from materials of Examples 5a-11a

| Example No. | Contact angle (°) | 100% modulus (N/mm²) |
|---|---|---|
| Comparative Example 3a | 21 | 2.3 |
| Example 4a | 22 | 7.9 |
| Example 5a | 26 | 4.1 |

Inventive Examples 4a and 5a differ in that, in comparison to comparative Example 3a, some of the polycarbonate diol Desmophen C2200 was replaced by the polycarbonate diol essential to the invention. In the form of a coating, the materials have hydrophilic properties similar to those of comparative Example 3a. The 100% moduli are all higher than that of comparative Example 3a.

### Example 7a Comparative Example

This example describes the production of a comparative example of a solution of polyurethaneurea.

195.4 g of Desmophen C 2200, 40.0 g of LB 25 and 47.8 g of 4,4′-bis(isocyanato-cyclohexyl)methane (H₁₂MDI) were reacted at 110° C. to a constant NCO content of 2.2%. The mixture was allowed to cool and was diluted with 350.0 g of toluene and 200 g of isopropanol. At room temperature, a solution of 12.0 g of isophoronediamine in 100 g of 1-methoxypropan-2-ol was added. After the end of the increase in molar weight, and the attainment of the desired viscosity range, stirring was continued for 4 hours in order to block the residual isocyanate content with isopropanol. This gave 945 g of a 31.6% strength solution of polyurethaneurea in toluene/isopropanol/1-methoxypropan-2-ol with a viscosity of 19,300 mPas at 22° C.

### Example 8a (Inventive)

97.5 g of Desmophen C 2200, 48.9 g of polycarbonate diol of Example 1, 40.0 g of LB 25 and 47.8 g of 4,4′-bis(isocyanatocyclohexyl)methane (H₁₂MDI) were reacted at 110° C. to a constant NCO content of 2.5%. The mixture was allowed to cool and was diluted with 320.0 g of toluene and 180 g of isopropanol. At room temperature, a solution of 11.2 g of isophoronediamine in 100 g of 1-methoxypropan-2-ol was added. After the end of the increase in molar weight, and the attainment of the desired viscosity range, stirring was continued for 5 hours at room temperature in order to block the residual isocyanate content with isopropanol. This gave 845.4 g of a 29.5% strength solution of polyurethaneurea in toluene/isopropanol/1-methoxypropan-2-ol with viscosity of 13,100 mPas at 22° C.

### Example 9a (Inventive)

146.8 g of Desmophen C 2200, 24.5 g of polycarbonate diol of Example 1, 40.0 g of LB 25 and 47.8 g of 4,4′-bis(isocyanatocyclohexyl)methane (H₁₂MDI) were reacted at 110° C. to a constant NCO content of 2.4%. The mixture was allowed to cool and was diluted with 320.0 g of toluene and 180 g of isopropanol. At room temperature, a solution of 11.5 g of isophoronediamine in 97.9 g of 1-methoxypropan-2-ol was added. After the end of the increase in molar weight, and the attainment of the desired viscosity range, stirring was continued for 22 hours at room temperature in order to block the residual isocyanate content with isopropanol. This gave 868.5 g of a 31.6% strength solution of polyurethaneurea in toluene/isopropanol/1-methoxypropan-2-ol with a viscosity of 32,800 mPas at 22° C.

### Example 10a (Inventive)

97.5 g of Desmophen C 2200, 25.6 g of polycarbonate diol of Example 2, 40.0 g of LB 25 and 47.8 g of 4,4′-bis(isocyanatocyclohexyl)methane (H₁₂MDI) were reacted at 110° C. to a constant NCO content of 3.0%. The mixture was allowed to cool and was diluted with 310.0 g of toluene and 170 g of isopropanol. At room temperature, a solution of 12.7 g of isophoronediamine in 100 g of 1-methoxypropan-2-ol was added. After the end of the increase in molar weight, and the attainment of the desired viscosity range, stirring was continued for 22 hours at room temperature in order to block the residual isocyanate content with isopropanol. This gave 803.6 g of a 28.2% strength solution of polyurethaneurea in toluene/isopropanol/1-methoxypropan-2-ol with a viscosity of 8600 mPas at 22° C.

### Example 11a (Inventive)

146.8 g of Desmophen C 2200, 12.8 g of polycarbonate diol of Example 2, 40.0 g of LB 25 and 47.8 g of 4,4′-bis(isocyanatocyclohexyl)methane (H₁₂MDI) were reacted at 110° C. to a constant NCO content of 2.5%. The mixture was allowed to cool and was diluted with 330.0 g of toluene and 185 g of isopropanol. At room temperature, a solution of 11.7 g of isophoronediamine in 100 g of 1-methoxypropan-2-ol was added. After the end of the increase in molar weight, and the attainment of the desired viscosity range, stirring was continued for 6 hours at room temperature in order to block the residual isocyanate content with isopropanol. This gave 874.1 g of a 30.0% strength solution of polyurethaneurea in toluene/isopropanol/1-methoxypropan-2-ol with a viscosity of 11,400 mPas at 22° C.

### Example 12a

### Contact Angles and 100% Moduli of Comparative Example 7a Versus Inventive Examples 8a-11a

### 1. Production of the Coatings for the Measurement of the Static Contact Angle

The coatings for the measurement of the static contact angle were produced on glass slides measuring 25×75 mm using a spin coater (RC5 Gyrset 5, Karl Süss, Garching, Germany). For this purpose, a slide was clamped on to the sample plate of the spin coater and covered homogeneously with about 2.5-3 g of organic 15% strength polyurethaneurea solution. All of the organic polyurethaneurea solutions were diluted to a polymer content of 15% using a solvent mixture of 65% by weight toluene and 35% by weight isopropanol. Rotation of the sample plate at 1300 revolutions per minute for 20 seconds gave a homogeneous coating, which was dried at 100° C. for 1 h and then at 50° C. for 24 h. The coated slides obtained were subjected directly to a contact angle measurement.

A static contact angle measurement was performed on the resulting coatings on the slides. Using the video contact angle measuring instrument OCA20 from Dataphysics, with computer-controlled injection, 10 drops of Millipore water were applied to the specimen, and their static wetting contact angle was measured. Beforehand, using an antistatic drier, the static charge (if present) on the sample surface was removed.

### 2. Production of the Coatings for the Measurement of the 100% Modulus

Films are produced on release paper using a 200 μm coating bar, and are dried at 100° C. for 15 minutes. Punched shapes are investigated in accordance with DIN 53504.

### 3. Results of Investigation

TABLE 2
Contact angles and 100% moduli of the
films of materials of Examples 7a-11a

| Example No. | Contact angle (°) | 100% modulus (N/mm²) |
|---|---|---|
| Comparative Example 7a | 14 | 2.7 |
| Example 8a | 19 | 5.6 |
| Example 9a | 22 | 3.5 |
| Example 10a | 26 | 6.2 |
| Example 11a | 15 | 3.9 |

Inventive Examples 8a to 11a differ in that, in comparison to comparative Example 7a, a part of the polycarbonate diol Desmophen C2200 was replaced by the polycarbonate diol essential to the invention. As a coating, the materials exhibit similarly hydrophilic properties to comparative Example 7a. The 100% moduli are all higher than that of comparative Example 7a.

### Polyurethaneurea Dispersions

### Example 3b (Comparative)

277.2 g of Desmophen C2200, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The mixture was heated to 110° C. After 16 hours, the theoretical NCO value of 2.4% was reached. The completed prepolymer was dissolved in 711 g of acetone at 50° C. and then at 40° C. a solution of 4.8 g of ethylenediamine in 16 g of water was metered in over the course of 10 minutes. The subsequent stirring time was 5 minutes. Subsequently, over the course of 15 minutes, dispersion was effected by addition of 590 g of water. The solvent was removed by distillation under reduced pressure. This gave a storage-stable polyurethaneurea dispersion having a solids content of 40.7% and an average particle size of 136 nm.

### Example 4b (Inventive)

138.6 g of Desmophen C2200, 69.3 g of polycarbonate diol from Example 1, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The mixture was heated to 110° C. After 75 minutes, the theoretical NCO value of 2.9% was reached. The completed prepolymer was dissolved in 700 g of acetone at 50° C. and then at 40° C. a solution of 4.8 g of ethylenediamine in 16 g of water was metered in over the course of 10 minutes. The subsequent stirring time was 5 minutes. Subsequently, over the course of 15 minutes, dispersion was effected by addition of 530 g of water. The solvent was removed by distillation under reduced pressure. This gave a storage-stable polyurethaneurea dispersion having a solids content of 38.4% and an average particle size of 143 nm.

### Example 5b (Inventive)

208 g of Desmophen C2200, 34.7 g of polycarbonate diol from Example 1, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The mixture was heated to 110° C. After 16 hours, the theoretical NCO value of 2.6% was reached. The completed prepolymer was dissolved in 700 g of acetone at 50° C. and then at 40° C. a solution of 4.8 g of ethylenediamine in 16 g of water was metered in over the course of 10 minutes. The subsequent stirring time was 5 minutes. Subsequently, over the course of 15 minutes, dispersion was effected by addition of 550 g of water. The solvent was removed by distillation under reduced pressure. This gave a storage-stable polyurethaneurea dispersion having a solids content of 43.0% and an average particle size of 115 nm.

### Example 6b (Inventive)

138.6 g of Desmophen C2200, 36.2 g of polycarbonate diol from Example 2, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The mixture was heated to 110° C. After 14.5 hours, the theoretical NCO value of 3.3% was reached. The completed prepolymer was dissolved in 670 g of acetone at 50° C. and then at 40° C. a solution of 4.8 g of ethylenediamine in 16 g of water was metered in over the course of 10 minutes. The subsequent stirring time was 5 minutes. Subsequently, over the course of 15 minutes, dispersion was effected by addition of 500 g of water. The solvent was removed by distillation under reduced pressure. This gave a storage-stable polyurethaneurea dispersion having a solids content of 38.6% and an average particle size of 147 nm.

### Example 7b (Inventive)

208 g of Desmophen C2200, 18.1 g of polycarbonate diol from Example 2, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The mixture was heated to 110° C. After 17 hours, the theoretical NCO value of 2.7% was reached. The completed prepolymer was dissolved in 700 g of acetone at 50° C. and then at 40° C. a solution of 4.8 g of ethylenediamine in 16 g of water was metered in over the course of 10 minutes. The subsequent stirring time was 5 minutes. Subsequently, over the course of 15 minutes, dispersion was effected by addition of 550 g of water. The solvent was removed by distillation under reduced pressure. This gave a storage-stable polyurethaneurea dispersion having a solids content of 39.7% and an average particle size of 122 nm.

### Example 8b

### Contact Angles and 100% Moduli of Comparative Example 3b Versus Inventive Examples 4b-7b

### 1. Production of the Coatings for the Measurement of the Static Contact Angle

The coatings for the measurement of the static contact angle were produced on glass slides measuring 25×75 mm using a spin coater (RC5 Gyrset 5, Karl Süss, Garching, Germany). For this purpose, a slide was clamped on to the sample plate of the spin coater and covered homogeneously with about 2.5-3 g of aqueous undiluted polyurethaneurea dispersion. Rotation of the sample plate at 1300 revolutions per minute for 20 seconds gave a homogeneous coating, which was dried at 100° C. for 15 min and then at 50° C. for 24 h. The coated slides obtained were subjected directly to a contact angle measurement.

A static contact angle measurement is performed on the resulting coatings on the slides. Using the video contact angle measuring instrument OCA20 from Dataphysics, with computer-controlled injection, 10 drops of Millipore water are applied to the specimen, and their static wetting contact angle is measured. Beforehand, using an antistatic drier, the static charge (if present) on the sample surface is removed.

### 2. Production of the Coatings for the Measurement of the 100% Modulus

The coatings were applied to release paper using a 200 μm coating bar. Prior to film production, the aqueous dispersions are admixed with 2% by weight of a thickener (Borchi Gel A LA, Brochers, Langenfeld, Germany) and homogenized by stirring at RT for 30 minutes. The wet films were dried at 100° C. for 15 minutes.

Punched shapes were investigated in accordance with DIN 53504.

### 3. Results of Investigation

TABLE 1
Contact angles and 100% moduli of the
films of materials of Examples 3b-7b

| Example No. | Contact angle (°) | 100% modulus (N/mm²) |
|---|---|---|
| Comparative Example 3b | 10 | 2.6 |
| Example 4b | 16 | 9.4 |
| Example 5b | <10 | 3.8 |
| Example 6b | 22 | 8.2 |
| Example 7b | 16 | 3.3 |

Inventive Examples 4b to 7b differ in that, in comparison to comparative Example 4b, a part of the polycarbonate diol Desmophen C2200 was replaced by a new inventive polycarbonate diol. As a coating, the materials exhibit similarly hydrophilic properties to comparative Example 3b, always smaller contact angles than 30°. The 100% moduli are all higher than that of comparative Example 3b.

### Example 9b (Comparative Example)

277.2 g of Desmophen C 1200, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 75 minutes the theoretical NCO value of 2.4% was attained. The completed prepolymer was dissolved in 711 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 590 g of water. This was followed by removal of the solvent by distillation under reduced pressure. This gave a storage-stable polyurethaneurea dispersion having a solids content of 39.9% and an average particle size of 169 nm.

### Example 10b (Inventive)

208.0 g of Desmophen C 1200, 34.7 g of polycarbonate diol from Example 1, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 2 hours 40 minutes the theoretical NCO value of 2.6% was attained. The completed prepolymer was dissolved in 700 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 530 g of water. This was followed by removal of the solvent by distillation under reduced pressure. This gave a storage-stable polyurethaneurea dispersion having a solids content of 40.8% and an average particle size of 132 nm.

### Example 11b (Inventive)

138.6 g of Desmophen C 1200, 69.3 g of polycarbonate diol from Example 1, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 2.5 hours the theoretical NCO value of 2.9% was attained. The completed prepolymer was dissolved in 700 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 530 g of water. This was followed by removal of the solvent by distillation under reduced pressure. This gave a storage-stable polyurethaneurea dispersion having a solids content of 40.2% and an average particle size of 132 nm.

### Example 12b (Inventive)

138.6 g of Desmophen C 1200, 36.2 g of polycarbonate diol from Example 2, 33.1 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 2.5 hours the theoretical NCO value of 3.2% was attained. The completed prepolymer was dissolved in 670 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 500 g of water. This was followed by removal of the solvent by distillation under reduced pressure. This gave a storage-stable polyurethaneurea dispersion having a solids content of 39.7% and an average particle size of 151 nm.

### Example 13b

### Contact Angles and 100% Moduli of Comparative Example 9b Against Inventive Examples 10b-12b

### 1. Production of the Coatings for the Measurement of the Static Contact Angle

The coatings for the measurement of the static contact angle were produced on glass slides measuring 25×75 mm using a spin coater (RC5 Gyrset 5, Karl Süss, Garching, Germany). For this purpose, a slide was clamped on to the sample plate of the spin coater and covered homogeneously with about 2.5-3 g of aqueous undiluted polyurethaneurea dispersion. Rotation of the sample plate at 1300 revolutions per minute for 20 seconds gave a homogeneous coating, which was dried at 100° C. for 15 minutes and then at 50° C. for 24 h. The coated slides obtained were subjected directly to a contact angle measurement.

A static contact angle measurement is performed on the resulting coatings on the slides. Using the video contact angle measuring instrument OCA20 from Dataphysics, with computer-controlled injection, 10 drops of Millipore water are applied to the specimen, and their static wetting contact angle is measured. Beforehand, using an antistatic drier, the static charge (if present) on the sample surface is removed.

### 2. Production of the Coatings for the Measurement of the 100% Modulus

The coatings were applied to release paper using a 200 μm doctor blade. Prior to film production, the aqueous dispersions are admixed with 2% by weight of a thickener (Borchi Gel A LA, Borchers, Langenfeld, Germany) and homogenized by stirring at RT for 30 minutes. The wet films were dried at 100° C. for 15 minutes.

Punched shapes were investigated in accordance with DIN 53504.

### 3. Results of Investigation

TABLE 2
Contact angles and 100% moduli of the
films from materials of Examples 9b-12b

| Example No. | Contact angle (°) | 100% modulus (N/mm²) |
|---|---|---|
| Comparative Example 9b | 14 | 1.6 |
| Example 10b | 23 | 2.7 |
| Example 11b | 22 | 4.7 |
| Example 12b | 26 | 3.9 |

Inventive Examples 10b to 12b differ in that, in comparison to comparative Example 9b, a part of the polycarbonate diol Desmophen C1200 was replaced by a new inventive polycarbonate diol. As a coating, the materials exhibit similarly hydrophilic properties to comparative Example 9b, always smaller contact angles than 30°. The 100% moduli are all higher than that of comparative Example 9b.

### Example 14b Comparative Example

269.8 g of Desmophen C 2200, 49.7 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 21 hours the theoretical NCO value of 2.4% was attained. The completed prepolymer was dissolved in 711 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 590 g of water. This was followed by removal of the solvent by distillation under reduced pressure. This gave a storage-stable polyurethaneurea dispersion having a solids content of 41.3% and an average particle size of 109 nm.

### Example 15b (Inventive)

135 g of Desmophen C 2200, 67.5 g of polycarbonate diol from Example 1, 49.7 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 17.5 hours the theoretical NCO value of 2.5% was attained. The completed prepolymer was dissolved in 650 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 520 g of water. This was followed by removal of the solvent by distillation under reduced pressure. This gave a storage-stable polyurethaneurea dispersion having a solids content of 39.8% and an average particle size of 80 nm.

### Example 16b (Inventive)

202.4 g of Desmophen C 2200, 33.7 g of polycarbonate diol from Example 1, 49.7 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 17 hours the theoretical NCO value of 2.5% was attained. The completed prepolymer was dissolved in 650 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 540 g of water. This was followed by removal of the solvent by distillation under reduced pressure. This gave a storage-stable polyurethaneurea dispersion having a solids content of 41.3% and an average particle size of 85 nm.

### Example 17b (Inventive)

202.6 g of Desmophen C 2200, 17.7 g of polycarbonate diol from Example 2, 49.7 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 17 hours the theoretical NCO value of 2.6% was attained. The completed prepolymer was dissolved in 670 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 550 g of water. This was followed by removal of the solvent by distillation under reduced pressure. This gave a storage-stable polyurethaneurea dispersion having a solids content of 41.4% and an average particle size of 85 nm.

### Example 18b (Inventive)

135 g of Desmophen C 2200, 35.3 g of polycarbonate diol from Example 2, 49.7 g of Polyether LB 25 and 6.7 g of neopentyl glycol were introduced at 65° C. and homogenized by stirring for 5 minutes. This mixture was admixed at 65° C. over the course of 1 minute first with 71.3 g of 4,4′-bis(isocyanatocyclohexyl)methane (H₁₂MDI) and thereafter with 11.9 g of isophorone diisocyanate. The batch was heated to 110° C. After 17.5 hours the theoretical NCO value of 3.1% was attained. The completed prepolymer was dissolved in 620 g of acetone at 50° C., and subsequently, at 40° C., a solution of 4.8 g of ethylenediamine in 16 g of water was metered in over the course of 10 minutes. The subsequent stirring time was 5 minutes. Subsequently, over the course of 15 minutes, dispersion was effected by addition of 490 g of water. This was followed by removal of the solvent by distillation under reduced pressure. This gave a storage-stable polyurethaneurea dispersion having a solids content of 39.2% and an average particle size of 79 nm.

### Example 19b

### Contact Angles and 100% Moduli of Comparative Example 14b Against Inventive Examples 15b-18b

### 1. Production of the Coatings for the Measurement of the Static Contact Angle

The coatings for the measurement of the static contact angle were produced on glass slides measuring 25×75 mm using a spin coater (RC5 Gyrset 5, Karl Süss, Garching, Germany). For this purpose, a slide was clamped on to the sample plate of the spin coater and covered homogeneously with about 2.5-3 g of aqueous undiluted polyurethane dispersion. Rotation of the sample plate at 1300 revolutions per minute for 20 seconds gave a homogeneous coating, which was dried at 100° C. for 15 minutes and then at 50° C. for 24 h. The coated slides obtained were subjected directly to a contact angle measurement.

A static contact angle measurement is performed on the resulting coatings on the slides. Using the video contact angle measuring instrument OCA20 from Dataphysics, with computer-controlled injection, 10 drops of Millipore water are applied to the specimen, and their static wetting contact angle is measured. Beforehand, using an antistatic drier, the static charge (if present) on the sample surface is removed.

### 2. Production of the Coatings for the Measurement of the 100% Modulus

The coatings were applied to release paper using a 200 μm doctor blade. Prior to film production, the aqueous dispersions are admixed with 2% by weight of a thickener (Borchi Gel A LA, Borchers, Langenfeld, Germany) and homogenized by stirring at RT for 30 minutes. The wet films were dried at 100° C. for 15 minutes.

Punched shapes were investigated in accordance with DIN 53504.

### 3. Results of Investigation

TABLE 3
Contact angles and 100% moduli of the films
from materials of Examples 14b-18b

| Example No. | Contact angle (°) | 100% modulus (N/mm²) |
|---|---|---|
| Comparative Example 14b | 11 | 1.9 |
| Example 15b | 13 | 5.0 |
| Example 16b | <10 | 2.8 |
| Example 17b | <10 | 2.9 |
| Example 18b | 17 | 4.4 |

Inventive Examples 15b to 18b differ in that, in comparison to comparative Example 14b, a part of the polycarbonate diol Desmophen C2200 was replaced by a new polycarbonate diol according to the invention. As a coating, the materials exhibit similarly hydrophilic properties to comparative Example 14b, always smaller contact angles than 30°. The 100% moduli are all higher than that of comparative Example 14b.

1-15. (canceled)

16. Polyurethaneurea, which is terminated with at least one copolymer unit of polyethylene oxide and polypropylene oxide, characterized in that the polyurethaneurea has at least one structural unit of the formula (I) 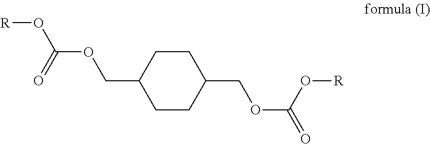
which is linked to the polymer chain by at least one bond R.

17. Polyurethaneurea according to claim 16, characterized in that it is free from ionic or ionogenic groups.

18. Polyurethaneurea according to claim 16, characterized in that it is based on a polycarbonate polyol component, which preferably has an average hydroxyl functionality of 1.7 to 2.3.

19. Polyurethaneurea according to claim 18, characterized in that polycarbonate polyol component comprises polycarbonate polyols a1) which are obtainable by reacting carbonic acid derivatives with difunctional alcohols of the formula (II)

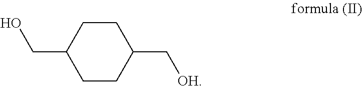

20. Polyurethaneurea according to claim 19, characterized in that polycarbonate polyol component comprises, in addition to the polycarbonate polyols a1), further polycarbonate polyols a2).

21. Polyurethaneurea according to claim 20, characterized in that the polycarbonate polyols a2) are compounds having an average hydroxyl functionality of 1.7 to 2.3 and a molecular weight, as determined by the OH number of 400 to 6000 g/mol, based on hexane-1,6-diol, butane-1,4-diol or mixtures thereof.

22. Polyurethaneurea according to claim 16, characterized in that the copolymer unit of polyethylene oxide and polypropylene oxide that is used for termination is based on a monohydroxy-functional mixed polyalkylene oxide polyether of at least 40 mol % ethylene oxide units and not more than 60 mol % propylene oxide units based on the total fraction of alkylene oxide units with a number-average molecular weight of 500 g/mol to 5000 g/mol.

23. Polyurethaneurea according to claim 16, characterized in that it is present in dissolved form.

24. Polyurethaneurea according to claim 23, characterized in that it is in solution in dimethylformamide, N-methylacetamide, tetramethylurea, N-methylpyrrolidone, toluene, linear and cyclic esters, ethers, ketones and/or alcohol and preferably in mixtures of toluene and ethanol, n-propanol, isopropanol and/or 1-methoxy-2-propanol.

25. Polyurethaneurea according to claim 16, characterized in that it is present in dispersed form.

26. Polyurethaneurea according to claim 25, characterized in that it is in dispersion in water.

27. Polyurethaneurea according to claim 16, characterized in that it comprises at least one active pharmacological ingredient.

28. Use of a polyurethaneurea according to claim 16 for producing a coating on a substrate.

29. Use according to claim 28, characterized in that the substrate is a medical device, more particularly a stent or a catheter.

30. Process for preparing a polyurethaneurea according to claim 16, in which a polycarbonate polyol component a), at least one polyisocyanate component b), at least one polyoxyalkylene ether component c), at least one diamine and/or amino alcohol component d) and, if desired, a further polyol component are reacted with one another.
